(19) United States
(12) Patent Application Publication
(10) Pub. No.: US 2005/0208504 A1
(43) Pub. Date: Sep. 22, 2005

(54) Method and system for testing feature-extractability of high-density microarrays using an embedded pattern block

(76) Inventors: Srinka Ghosh, San Francisco, CA (US); Eric M. Leproust, San Jose, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/803,510

(22) Filed: Mar. 17, 2004

(51) Int. Cl. C12Q 1/68; C12M 1/34

(52) U.S. Cl. 435/6; 435/287.2

(57) ABSTRACT

A method and system for monitoring the feature-extractability of microarrays by integrating control-feature blocks, or reference pattern blocks within a microarrray. The embedded control features comprise an array of pattern blocks, or reference pattern, in which each pattern block is composed of a set of microarray features arranged in a specific pattern of low-intensity and high-intensity features. Positive control features are designed to generate high-intensity signals following exposure of the microarray to a sample solution, and negative control features are designed to generate no signal or a low-intensity signal. The pattern blocks may be visually inspected to determine the feature extractability of a microarray prior to undertaking full, automated feature extraction, or may select a feature-extraction method based on an analysis of the reference pattern.

### BACKGROUND OF THE INVENTION

Embodiments of the present invention relates to a reference pattern used to facilitate feature extractability of microarrays of low, intermediate, and high densities. In high density arrays having small inter-feature spacings, the background regions for features may not be easily distinguished from neighboring feature-containing regions, leading to difficulties in applying feature-extraction methods that rely on background-intensity determination.

In order to facilitate discussion of the present invention, a general background for microarrays is provided, below. In the following discussion, the terms “microarray,” “molecular array,” and “array” are used interchangeably. The terms “microarray” and “molecular array” are well known and well understood in the scientific community. As discussed below, a microarray is a precisely manufactured tool which may be used in research, diagnostic testing, or various other analytical techniques.

Array technologies have gained prominence in biological research and in diagnostics. Currently, microarray techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any-type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations A, T, C, and G for the nucleotide subunits that together compose the DNA polymer. For example, the oligomer 100 shown in FIG. 1 can be chemically represented as “ATCG.”

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction, or, in other words, the two strands are anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand. FIGS. 2A-B illustrate the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. Although deoxy-guanylate subunits of one strand are generally paired with deoxy-cytidilate subunits from the other strand, and deoxy-thymidilate subunits in one strand are generally paired with deoxy-adenylate subunits from the other strand, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contain nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for scanning. Biotin and avidin represent an example of a pair of chemical moieties that can be utilized for such steps.

Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

When a microarray is scanned, data may be collected as a two-dimensional digital image of the microarray, each pixel of which represents the intensity of phosphorescent, fluorescent, chemiluminescent, or radioactive emission from an area of the microarray corresponding to the pixel. A microarray data set may comprise a two-dimensional image or a list of numerical or alphanumerical pixel intensities, or any of many other computer-readable data sets. An initial series of steps employed in processing digital microarray images includes constructing a regular coordinate system for the digital image of the microarray by which the features within the digital image of the microarray can be indexed and located. For example, when the features are laid out in a periodic, rectilinear pattern, a rectilinear coordinate system is commonly constructed so that the positions of the centers of features lie as closely as possible to intersections between horizontal and vertical gridlines of the rectilinear coordinate system, alternatively, exactly half-way between a pair of adjacent horizontal and a pair of adjacent vertical grid lines. Then, regions of interest (“ROIs”) are computed, based on the initially estimated positions of the features in the coordinate grid, and centroids for the ROIs are computed in order to refine the positions of the features. Once the position of a feature is refined, feature pixels can be differentiated from background pixels within the ROI, and the signal corresponding to the feature can then be computed by integrating the intensity over the feature pixels.

A general trend in microarray manufacturing is to make microarrays of higher feature density in order to increase the number of probes interrogated per experiment. One approach for increasing microarray feature density is to proportionately decrease the feature and inter-feature dimensions. However, this approach is likely to impact the accuracy of signal intensities interrogated from high density arrays, since absolute feature size and the number of pixels associated with a feature may correlate with the signal-to-noise ratio of the system. For example, as the number of pixels allocated to detect signal intensities is decreased, the confidence of the signal intensity measurement may be lowered even though the average signal intensity may remain unchanged. Proportionally decreasing the feature and inter-feature dimensions may not be feasible due to technological limitations, and may lead to a relative decrease in the accuracy of measuring background intensities near features. For these and many other reasons, as the feature density of microarrays increases, the percentage of microarrays that can be analyzed using current automated feature-extraction techniques has been found to have substantially decreased. Designers and manufacturers of microarrays have therefore recognized the need for a method for determining whether or not intensity signals can be reliably extracted from a particular high-density microarray prior to employing automated feature-extraction methods when using a particular automated feature-extraction method.

### SUMMARY OF THE INVENTION

One embodiment of the present invention provides a method and system for evaluating the feature-extractability of high-density microarrays by integrating, control-feature blocks, or pattern blocks, within microarrays and using the pattern blocks to evaluate feature extractability. In a disclosed embodiment, control features are integrated within the design of high-density microarrays, including microarrays with features that are packed densely together in a hexagonal pattern. The embedded control features comprise an array of pattern blocks, or a reference pattern, in which each pattern block is composed of a set of microarray features arranged in a specific pattern of low-intensity and high-intensity features. The reference pattern can be embedded or replicated anywhere on the surface of a microarray. The pattern blocks may be visually inspected to determine the feature extractability of a microarray prior to undertaking full, automated feature extraction, or may select a feature-extraction method based on an analysis of the reference pattern.

### BRIEF DESCRIPTION OF THE DRAWINGS

FIG. 1 illustrates a short DNA polymer.

FIG. 2A shows hydrogen bonding between adenine and thymine bases of corresponding adenosine and thymidine subunits.

FIG. 2B shows hydrogen bonding between guanine and cytosine bases of corresponding guanosine and cytosine subunits.

FIG. 3 illustrates a short section of a DNA double helix.

FIGS. 4-7 illustrate the principle of array-based hybridization assays.

FIGS. 8A-B illustrate a low-density feature arrangement and a more recently developed, high-feature-density, or double-density, feature arrangement within microarrays.

FIGS. 9A-B illustrate an initial coordinate grid superimposed over the feature arrangements illustrated in FIGS. 8A-B.

FIGS. 10A-B illustrate the construction of various types of ROIs around an initial feature position determined from an initial coordinate grid calculated for a microarray.

FIG. 11 is a general representation of a high-density microarray with disk-shaped features having inter-feature distances less than feature diameters, one of many different possible types of high-density microarrays.

FIGS. 12A-B illustrate a problem with local background-signal estimation that arises with high-feature-densities.

FIG. 13 illustrates a scanned image of a hypothetical double-density microarray, which is used to computationally determine feature signal intensities arising from feature ROIs.

FIGS. 14A-B illustrate the effect of neighboring high-intensity features on the displacement of the computed center for the low-intensity, central feature of a subregion.

FIG. 15A illustrates the positioning of a set of reference features incorporated within a high-density microarray.

FIG. 15B illustrates one preferred embodiment involving the placement of the two-dimensional reference pattern at two corners of a microarray.

FIGS. 16A-B illustrate the design for a two-dimensional reference pattern or image.

FIG. 17A illustrates a pattern block selected from the high-intensity central feature rows 0-2 with two high-intensity neighboring features and a pattern block selected from the low-intensity central feature rows 3-5 with two high-intensity neighboring features with an identical orientation to the central feature.

FIG. 17B illustrates another example of a complementary pair of pattern blocks.

FIG. 18 illustrates a kit for determining the existence of a feature extractability problem resulting from displacements of computed feature positions during a manufacturing process.

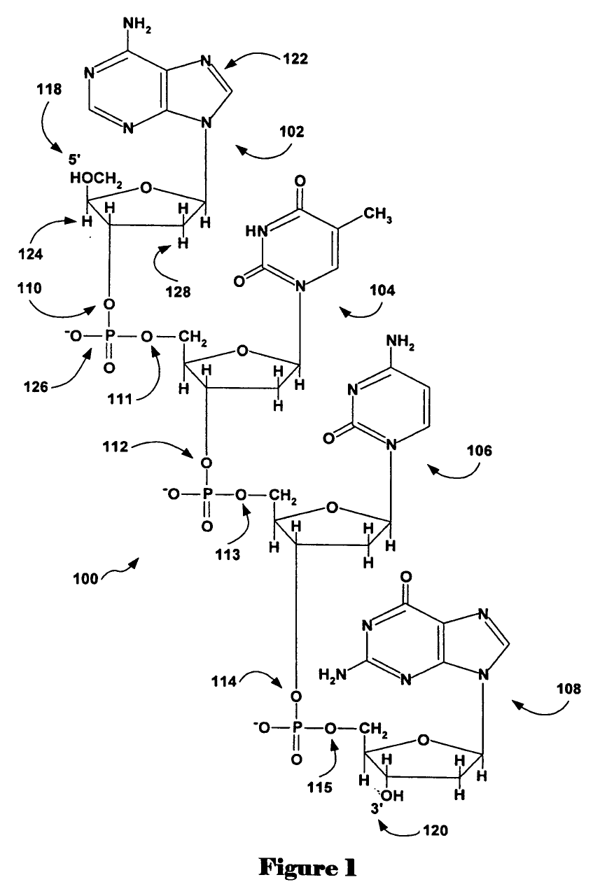

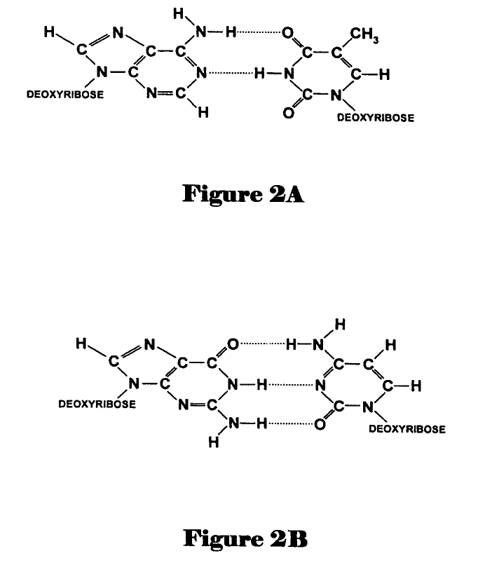

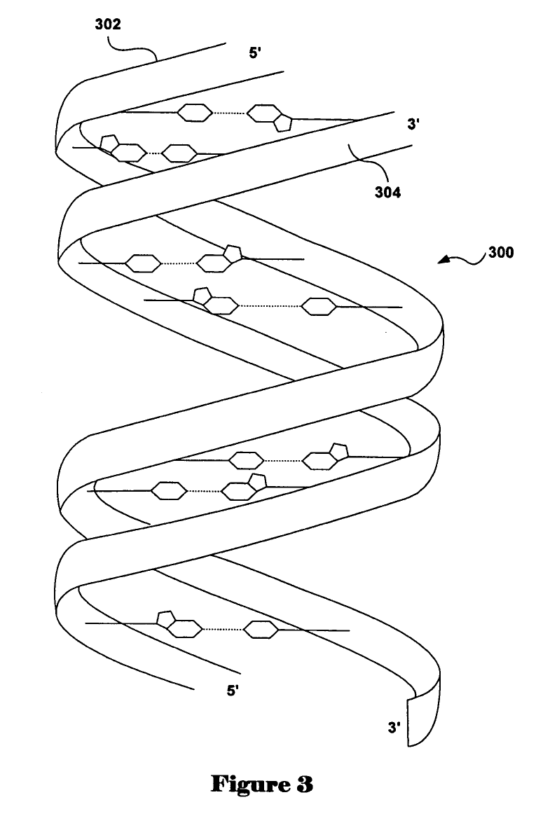

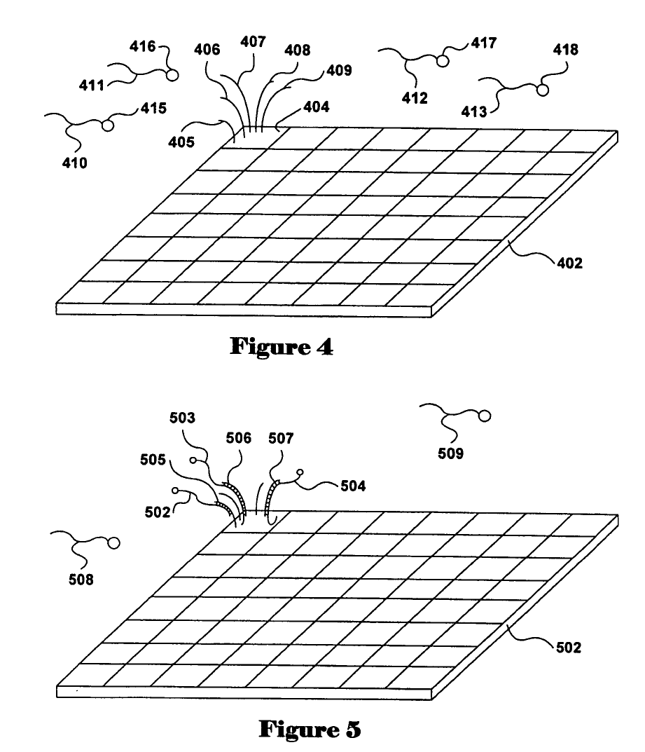

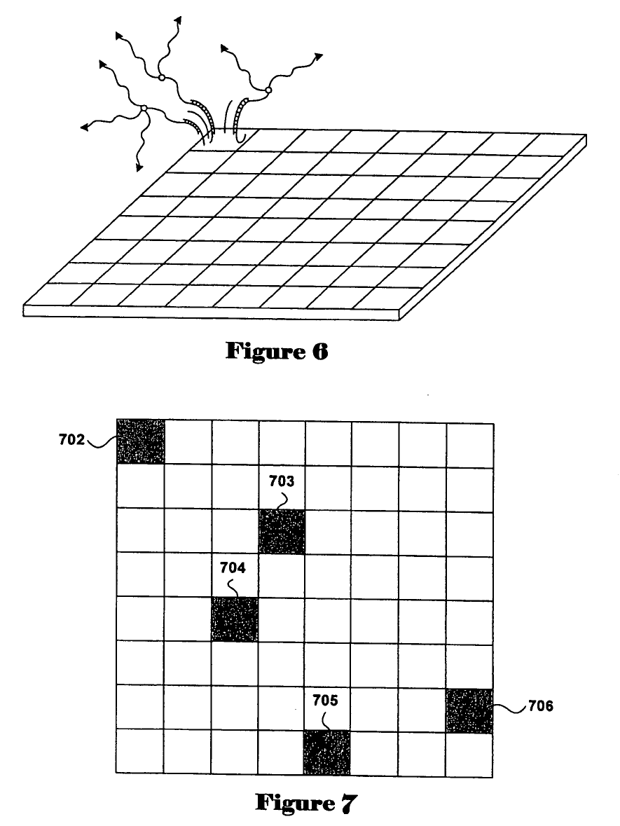

### DETAILED DESCRIPTION OF THE INVENTION

One embodiment of the present invention is directed to a method and system for ascertaining the feature-extractability of a high-density microarray by integrating, within the microarray, a two-dimensional reference pattern. In an embodiment described below, the reference pattern includes hexagonally packed positive and negative control features. Positive control features are designed to generate high-intensity signals following exposure of the microarray to a sample solution, and negative control features are designed to generate no signal or a low intensity signal. The embedded calibration device comprises a set of pattern blocks, each pattern block comprising a number of microarray features arranged in a specific pattern of low-intensity and high-intensity features, which are positioned at known locations on the microarray. In one embodiment of the present invention, the reference patterns are located at one or more corners of the microarray. The pattern blocks can be visually inspected to determine whether a particular high-density microarray is amenable to automated feature extraction. In alternative embodiments, an automated reference-pattern-checking subsystem may determine the feature extractability of a microarray prior to undertaking full, automated feature extraction, or may select a feature extraction method based on an analysis of the reference pattern.

The embodiments of the present invention can be implemented to detect centroid-displacement artifacts arising from differences in the intensities of adjacent features, irregularities in adjacent feature sizes, misalignment of adjacent feature positions, and other such phenomena. The following discussion includes two subsections, a first subsection including additional information about molecular arrays, and a second subsection describing embodiments of the present invention with reference to FIGS. 10-17.

### Additional Information About Microarrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Inter-feature areas are typically, but not necessarily, present. Inter-feature areas generally do not carry probe molecules. Such inter-feature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

A molecular array is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in published U.S. patent applications 20030160183A1, 20020160369A1, 20040023224A1, and 20040021055A, as well as U.S. Pat. No. 6,406,849. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, and elsewhere.

A result obtained from reading an array, followed by application of a method of the present invention, may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based molecular array, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a molecular array by an optical scanning device or radiometric scanning device generally produces an image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of molecular-array data can produce detailed chemical and biological analyses, disease diagnoses, and other information that can be stored in a computer-readable medium, transferred to an intercommunicating entity via electronic signals, printed in a human-readable format, or otherwise made available for further use.

### Embodiments of the Present Invention

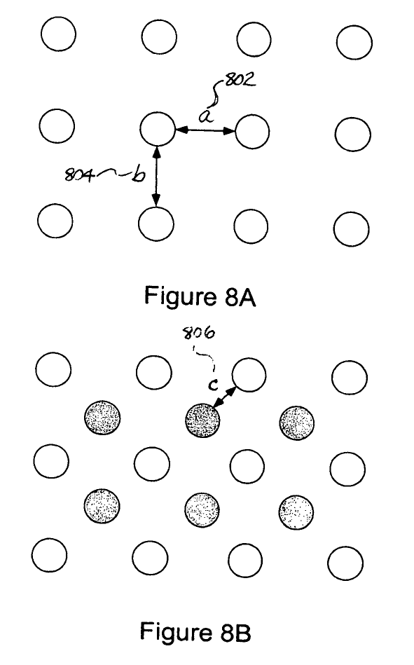

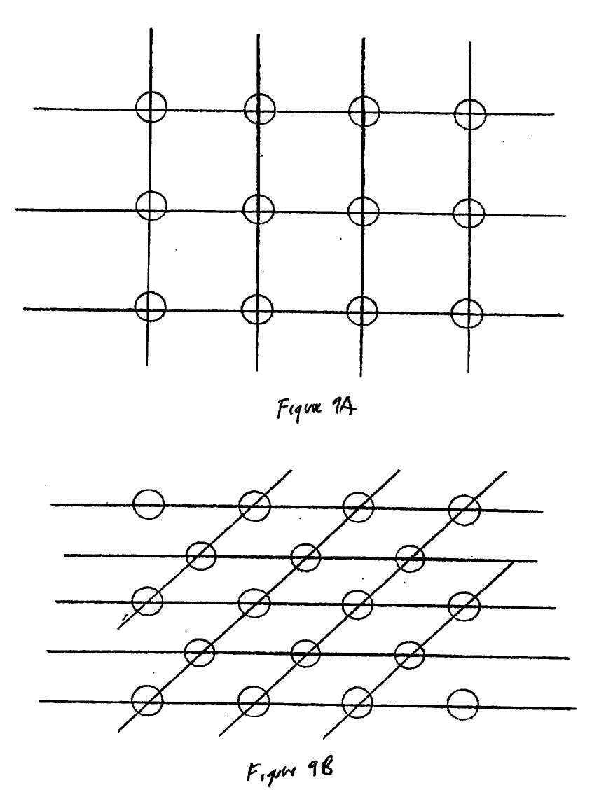

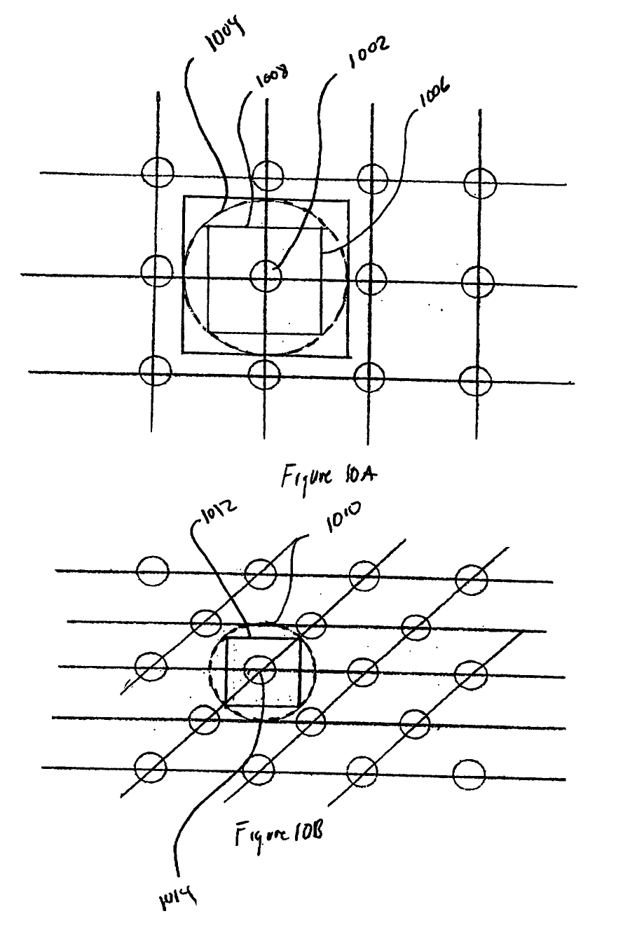

FIGS. 8A-B illustrate a low-density feature arrangement and a more recently developed, high-feature-density, or double-density, feature arrangement within microarrays. In both FIGS. 8A-B, a very small region of the surface of a microarray is illustrated. As can be seen by comparing FIG. 8A to FIG. 8B, the double-density-microarray feature arrangement doubles, or nearly doubles, the number of features within a given area of the microarray by packing the features together more closely. In the arrangements of features illustrated in FIGS. 8A-B, if the minimum distance between adjacent features is a 802 in the horizontal direction and b 804 in the vertical direction for the low-feature-density arrangement, shown in FIG. 8A, then the minimum distance between adjacent features c 806 in the newer, high-feature-density arrangement shown in FIG. 8B is

$$\frac{\sqrt{( {a}^{2} + {b}^{2} )}}{2}$$

when the high-feature-density arrangement is obtained by adding features in rows offset by one-half of a grid spacing in both horizontal and vertical directions.

FIGS. 9A-B illustrate an initial coordinate grid superimposed over the feature arrangements illustrated in FIGS. 8A-B. Again, as described above, the initial coordinate grid allows each feature to be indexed, and allows for an ROI to be calculated for each feature within the digital image of a microarray. FIGS. 10A-B illustrate the construction of various types of ROIs around an initial feature position determined from an initial coordinate grid calculated for a microarray. As shown in FIG. 10A, various different ROIs can be calculated for a feature 1002 in a low-feature-density microarray. Similar ROIs can also be constructed for high-feature-density microarrays, as shown in 10B. Given that, in many embodiments, features are roughly disc-shaped, a natural form for an ROI is a large disc 1004 centered at the initially calculated position of the feature 1002. This disc-shaped ROI 1004 should be as large as possible, in order to include as many pixels as possible for statistical analysis of background intensities in the region surrounding the feature, but should not be greater than a size at which the ROI might encroach on adjacent features. In order to speed calculation of the ROIs for thousands, tens of thousands, or hundreds of thousands of features within a digital image of a microarray with features arranged in a rectilinear grid, it is more computationally efficient to compute square or rectangular ROIs, such as ROIs 1006 and 1008. As with disc-shaped ROIs, rectangular ROIs should be as large as possible, in area, in order to include a sufficient number of pixels for meaningful statistical analysis of background pixels surrounding a feature, but should not be so large as to begin to include pixels of adjacent features. Note, in FIG. 10B, that the ROIs 1010 and 1012 computed for a feature 1014 in a double-density arrangement is significantly smaller then the ROIs 1004, 1006 and 1008 computed for a feature and a low-feature-density arrangement.

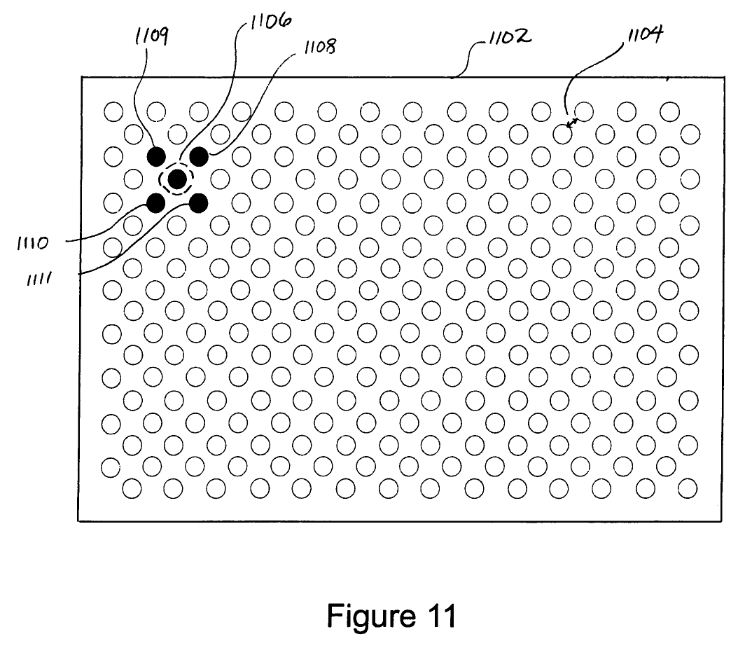

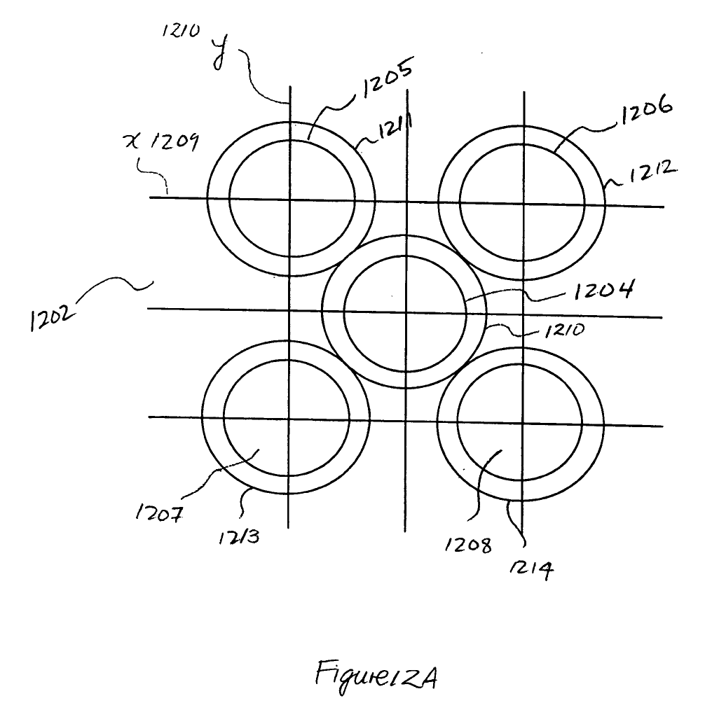

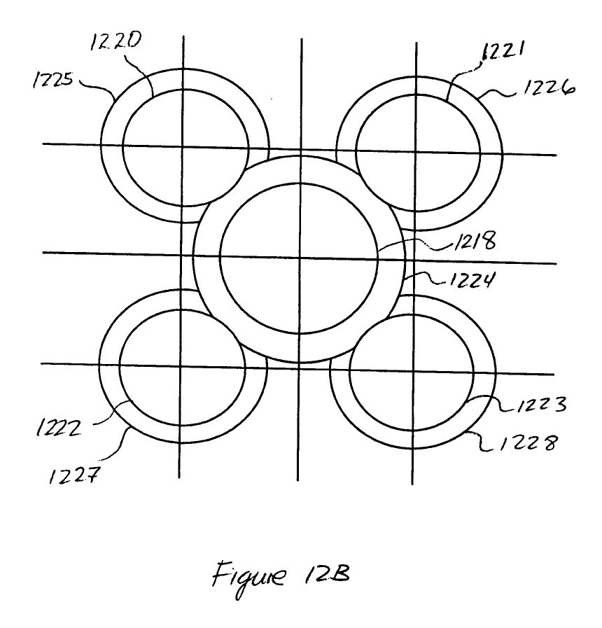

FIG. 11 is a general representation of a high-density microarray with disk-shaped features having inter-feature distances less than feature diameters, one of many different possible types of high-density microarrays that can be produced using ink-jet technology for printing features. A double-density microarray pattern 1102 is produced in which adjacent columns of features are positioned off-center with respect to one another in order to maximize the total space, and therefore, to minimize the inter-feature distance 1104 without decreasing feature size. This arrangement of features has the effect of decreasing inter-feature separations relative to feature dimensions. The present invention may be applied to various high-density microarray designs with many other types of feature arrangements involving relatively small inter-feature distances. The invention is described in the following figures with respect to a subset of microarray features with an arrangement of a central feature 1106 or feature of focus surrounded by four neighboring features 1108-1111.

FIGS. 12A-B illustrate a problem with local background-signal estimation that arises with high-feature-densities. FIG. 12A illustrates a small section 1202 of the high-density microarray of FIG. 11, in which the features are uniform in size and equidistantly positioned. A central feature 1204 is surrounded by four neighboring features 1205-1208 that are uniform in size and equidistantly positioned with respect to the center feature 1204. The initial coordinate grid with x 1209 and y 1210 coordinates allows each feature to be indexed, and allows for an ROI to be calculated for each feature within the digital image of a microarray. Various types of ROIs can be constructed around an initial feature position determined from an initial coordinate grid calculated for a microarray, including disk-shaped ROIs, square-shaped ROIs, and rectangular-shaped ROIs. In the example provided, each ROI of a feature is partitioned into sub-regions that comprise a central region referred to as an inner ROI, and an annulus region referred to as an outer ROI. In FIGS. 12A-B, the outer ROI can also represent a background annulus used to calculate background signal intensity for a given feature. It is apparent that as the inter-feature distances are decreased systematically, the probability that the background annulus of the central feature 1210 will overlap with the background annuli of neighboring features 1211-1214 increases. Because of background overlap, as the density of features placed on microarray substrates increases, local background-signal estimation techniques may begin to fail. Although it may be possible to decrease the width of background annuli in order to preclude overlapping, the background annuli cannot be arbitrarily decreased in size beyond a certain limit. There must be, for example, a minimum number of pixels within the background annulus in order to generate a statistically significant estimation of the intensity of pixels within the background region surrounding a feature.

FIG. 12B illustrates a small section 1202 of the high-density microarrays of FIG. 11, in which the features are not uniform in size. A central feature 1218 is surrounded by four neighboring features 1220-1223 that are smaller with respect to the center feature 1204. Differential sizes of features may arise during manufacturing processes in many ways, including printing-related error. In FIG. 12B, the background annulus 1224 of the larger center feature 1218 overlaps the ROIs of the four neighboring features 1220-1223 by extending beyond the inner boundaries of the background annulus 1225-1228 of the four features. This may significantly raise the background signal estimation for the center feature 1218 above the true, non-feature and non-ROI background-signal intensity level.

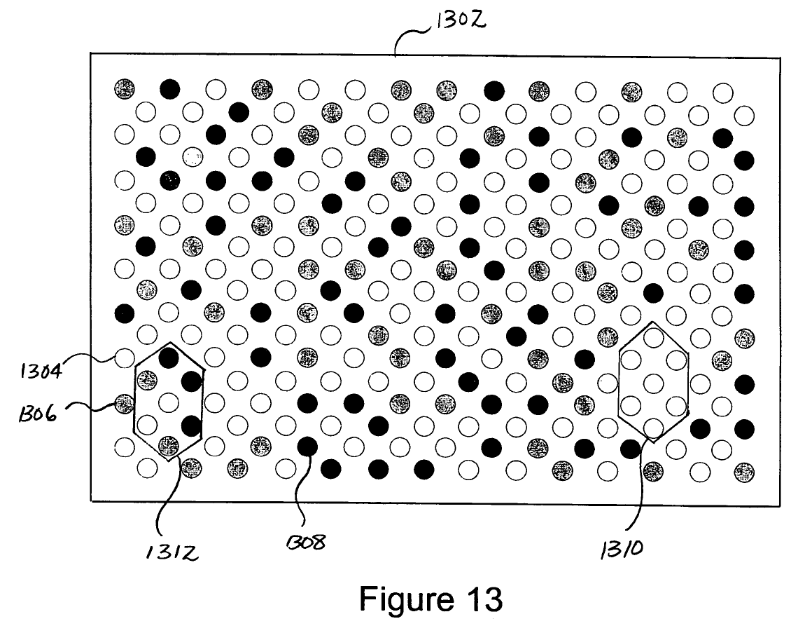

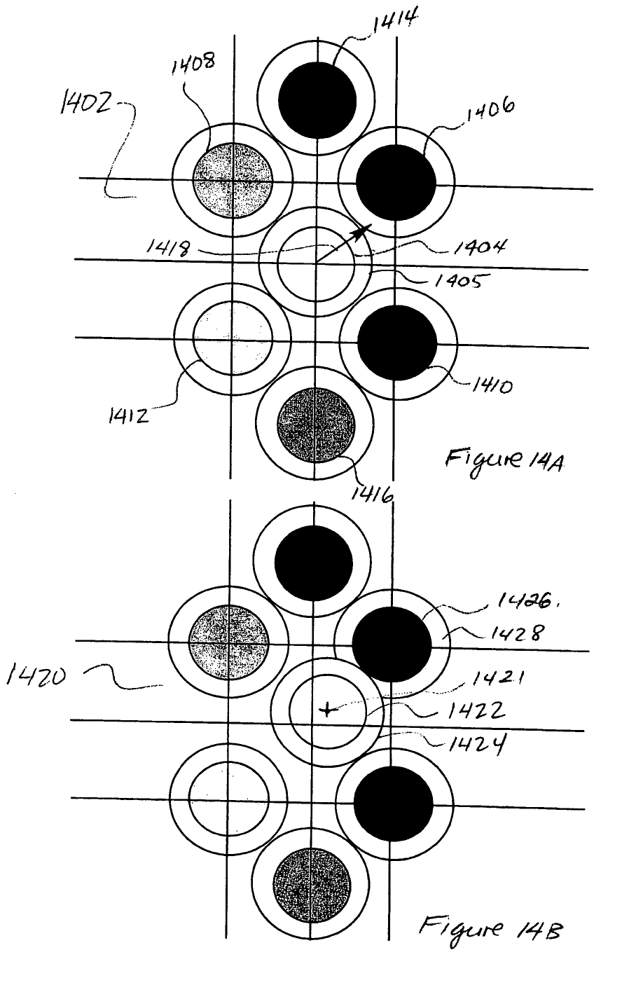

FIG. 13 illustrates a scanned image of a hypothetical double-density microarray, which is used to computationally determine feature signal intensities arising from feature ROIs. For example, a scanned image of a high-density microarray 1302 includes profiles of features with differential signal intensities shown for convenience as either white-colored disks, such as feature 1304 for very low-intensity, gray-colored disks, such as feature 1306 for intermediate-intensity, and black-colored disks, such as feature 1308 for very-high intensity. Problems in distinguishing boundaries among features can be exacerbated when features with high-signal intensities, such as feature 1308, are positioned next to features with low-signal intensities, such as feature 1304. When low-signal features are positioned near high-signal features, feature-extraction errors may result due to the displacement towards high-intensity features of the centers of low-intensity features, re-computed based on initial positions obtained by grid-finding methods and on pixel intensities within an ROI surrounding the features.

In FIG. 13, a sub-region of the microarray image 1310 includes a low-intensity, central feature surrounded by four low-intensity neighboring features, and another sub-region of the microarray image 1312 includes a low-intensity, central feature surrounded by four neighboring features comprising three high-intensity features and one low-intensity feature. FIGS. 14A-B illustrate the effect of neighboring high-intensity features on the displacement of the computed center for the low-intensity, central feature of sub-region 1312 in FIG. 13. In FIG. 14A, sub-region 1312 is shown with an initially computed position of the central feature 1404 with a background annulus 1405. The central feature 1404 is surrounded by four closest neighbor features that include the northeast adjacent feature 1406, the northwest adjacent feature 1408, the southeast adjacent feature 1410, and the southwest adjacent feature 1412. The north feature 1414 and south feature 1416 are more distantly located than these four closest neighbor features 1406, 1408, 1410, and 1412, and less affect the displacement of the re-computed center of the central feature. Because the northeast feature 1406 and southeast feature 1410 have higher average pixel intensities than the northwest feature 1408 and southwest feature 1412, and because the ROI of the central feature overlaps the ROIs of the closest neighboring features, the re-computed center of the central feature is displaced towards the northeast feature 1406 and southeast feature 1410, as indicated by the large centroid-displacement vector 1418. In FIG. 14B, the sub-region of a microarray corresponding to the sub-region 1410 of FIG. 13 is shown after the displacement of the re-computed center of the central feature. The ROI of feature 1422 is shifted towards the northeast feature 1426 as a result of the asymmetrical distribution of high-intensity and low-intensity features about the central feature. Note that the background annulus 1424 of feature 1422 overlaps the background annulus 1428 of feature 1426. An overlap in the background annulus 1424 of the central feature 1422 and the ROI of the adjacent feature 1426 can substantially increase the calculated background intensity for the central feature by the inclusion of background pixels from the neighboring feature. In more severe cases, a feature centroid may be sufficiently displaced to result in inclusion of neighboring feature pixels in the feature's background or in the feature itself.

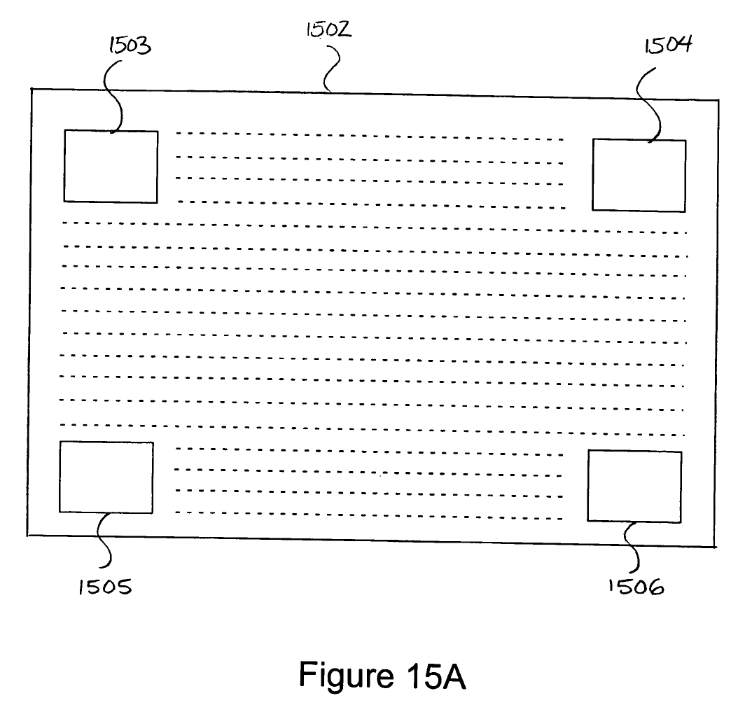

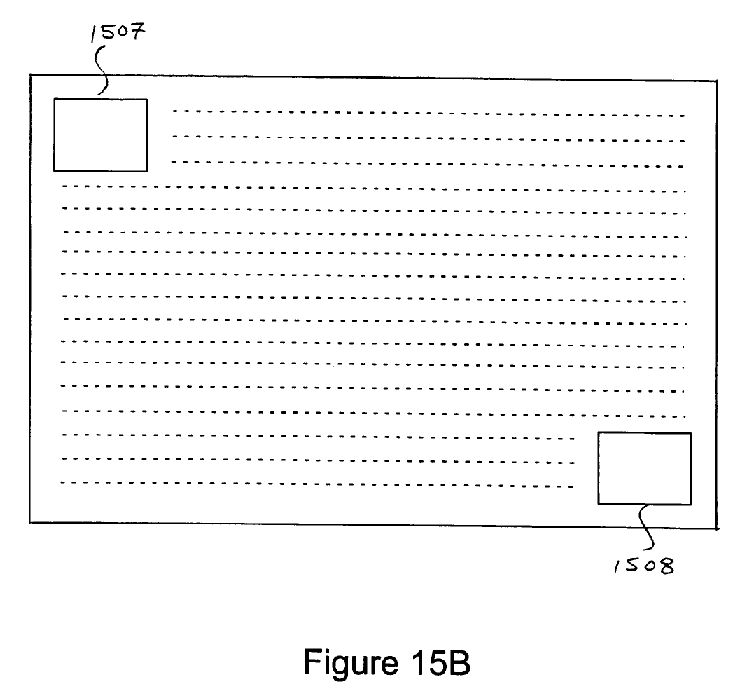

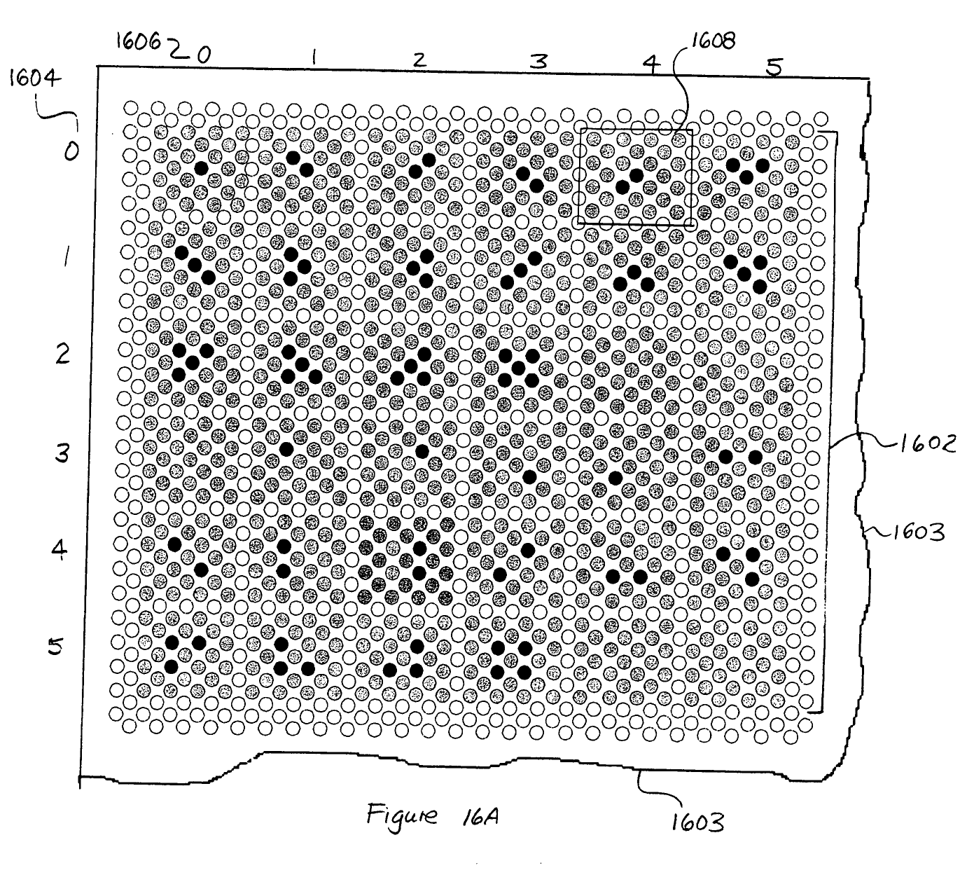

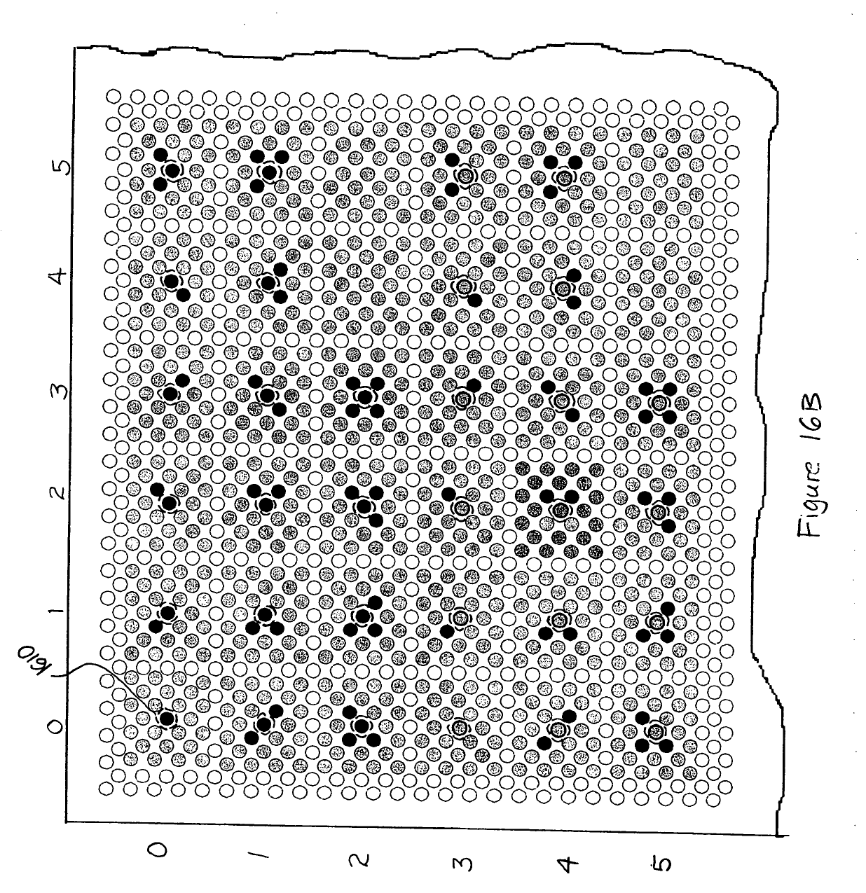

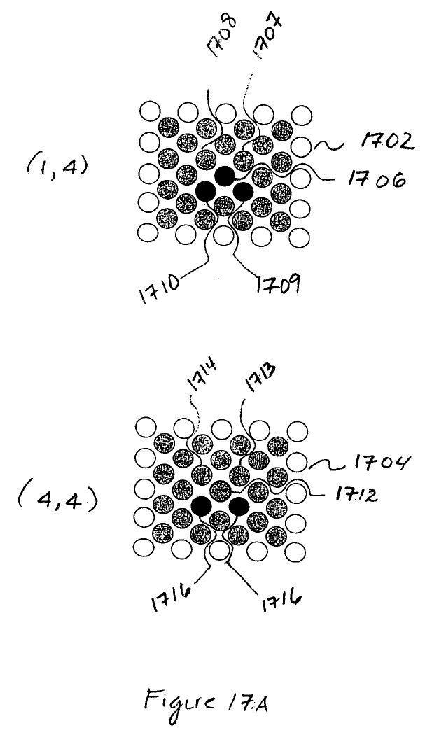

FIG. 15A illustrates the positioning of a set of reference features incorporated within a high-density microarray. In FIG. 15, a high-density microarray 1502 is shown with four corners: an upper left-hand corner 1503, an upper right-hand corner 1504, a lower left-hand corner 1505, and a lower right-hand corner 1506. Because high-density microarrays may be subject to signal-to-background calculation errors, a design for producing such microarrays that represents one embodiment of the present invention includes one or more reference patterns to allow for quickly determining the quality of a feature extraction performed on the microarray. A two-dimensional reference pattern, to be elaborated in FIGS. 16A-B provided below, can be designed to be an integral part of microarray feature composition, and positioned in strategic locations, such as in one or more corners of a microarray 1503-1506 that are particularly sensitive to variability introduced during manufacturing processes. The placement of a reference pattern can be repeated in all four corners of a microarray, as shown in FIG. 15A, so that redundancy in data and higher confidence in feature extractability can be achieved. FIG. 15B illustrates one preferred embodiment involving the placement of the two-dimensional reference pattern at two opposing corners of a microarray. When the surface area of the microarray is limited, the reference pattern can be positioned at two positions, rather than at four positions. A first reference pattern can be placed in corner 1507 and a second reference pattern can be placed in corner 1508, where the greatest process-oriented instabilities are observed. These instabilities are typically associated with the corners at which the feature-deposition process begins and ends, and where, due to starting and stopping of the print head used to deposit solutions for chemically synthesizing probe molecules, or for depository already synthesized probe molecules, feature sizes and spacings may be relatively non-uniform with respect to the sizes and spacings of features in the remaining portions of a microarray.

FIGS. 16A-B illustrate the design for a two-dimensional reference pattern or image. FIG. 16A shows a two-dimensional reference pattern 1602 positioned in the upper left-hand corner of a hypothetical microarray 1603. The two-dimensional reference pattern 1602 comprises a 6×6 pattern-block matrix, indexed by rows (0-5) 1604 and by columns (0-5) 1606. The reference pattern shown in FIG. 16 comprises 32 pixel-based pattern blocks, each rectangular pattern block, such as rectangular pattern block (0,0) 1608, comprising 25 hexagonally packed features. The two-dimensional reference pattern collectively represents all of the different possible arrangements of high-intensity and low-intensity nearest neighbor features about a central feature. Pattern blocks (2,4), (2,5), (5,4), and (5, 5) are not used, since there are only 32 possible nearest-neighbor arrangements, while there are 36 possible pattern blocks within the 6×6 pattern-block matrix. The pattern blocks are separated by rows and columns of low-intensity features to facilitate pattern-block recognition, and may facilitate automated methods that employ the two-dimensional reference pattern. In alternative embodiments, all 36 possible pattern blocks may be used by incorporation of redundant patterns.

FIG. 16B provides a pattern-block-centric representation of FIG. 16A. In FIG. 16B, unfilled circles, such as unfilled circle 1610, represent central, low intensity features. In FIG. 16B, the central feature of each pattern block is shown circumscribed by a dashed circle, such as dashed circle 1610. In the two-dimensional reference pattern, each pattern block in rows 0, 1, and 2 includes a high-intensity central feature, and each pattern block in rows 3, 4, and 5 includes a low-intensity central feature. Rows 0, 1, and 2 include pattern blocks representing all possible high and low-intensity feature patterns of the four nearest neighbors of a high-intensity central feature, and rows 3, 4, and 5 include pattern blocks representing all possible high and low-intensity feature patterns of the four nearest neighbors of a low-intensity central feature. For example, pattern blocks (0, 1), (0, 2), (0, 3), and (0, 4) include all possible arrangements of two high-intensity, nearest neighbor features about a high-intensity central feature.

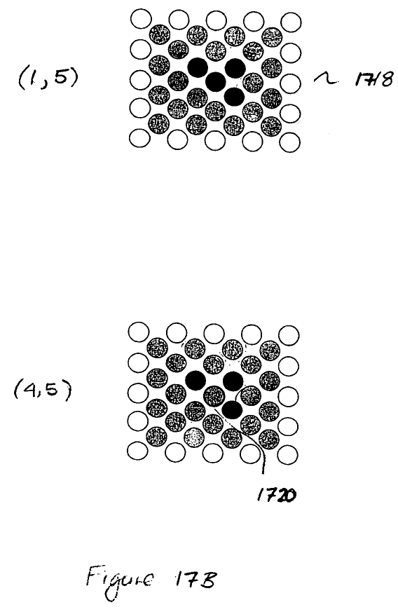

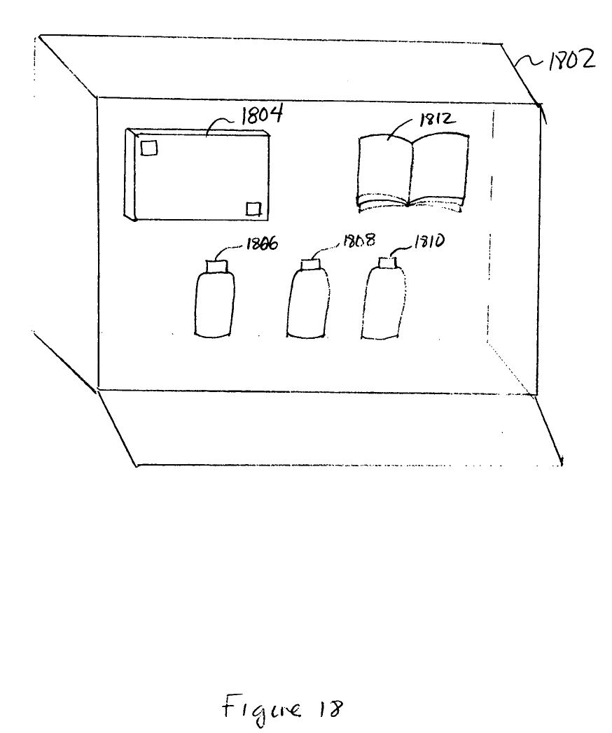

FIG. 17A illustrates a pattern block selected from the high-intensity central-feature rows 0-2 with two high-intensity neighboring features and a pattern block selected from the low-intensity central feature rows 3-5 with two high-intensity neighboring features with an identical orientation to that of the central feature. Pattern block (1, 4) 1702 includes a high-intensity central feature 1706 with two high-intensity nearest neighbors in the southeast and southwest positions 1709 and 1710, respectively, and two low-intensity nearest neighbors in the northeast and northwest positions 1707 and 1708, respectively. Pattern block (4, 4) 1704 includes a low-intensity central feature 1712 with two high-intensity nearest neighbors in the southeast and southwest positions 1715 and 1716, respectively, and two low-intensity nearest neighbors in the northeast and northwest positions 1713 and 1714, respectively. Thus, pattern blocks (1, 4) and (4, 4) together represent a complementary pair. Each pattern block in rows 0-2 having a high-intensity central feature has a complementary low-intensity central feature pattern block in one of rows 3-5. FIG. 17B illustrates another example of a complementary pair of pattern blocks. Pattern block (1,5) 1718 has a high-intensity central feature and three high-intensity nearest neighbors in the northwest, northeast, and southeast positions, and pattern block (2,0) 1720 has a low-intensity central feature and three high-intensity nearest neighbors in the northwest, northeast, and southeast positions.

FIG. 18 illustrates a kit for determining the existence of feature extractability problem resulting from displacements of computed feature positions during manufacturing process. The kit 1802 may include at least one microarray substrate 1804, one or more reference targets 1806, and one or more reagents needed for hybridizations 1808 and post-hybridization washes 1810. The microarray substrate comprises one or more sets of features arranged as a reference pattern and a number of features comprising probe molecules that can bind to sample target molecules, typically supplied by the user of the kit. The reference targets are molecules that can be added to biological samples as a spike-in, and which bind to the complementary probe molecules of the reference pattern features. The kit also includes written instructions 1812 for determining whether a feature extractability problem exists. The written instructions disclose a method for exposing the microarray substrate to reference targets. Feature extraction software may be used to facilitate feature extraction, and codes, such as bar codes, may be used to access one or more extraction methods stored on a local or a remote memory system. In another embodiment, in lieu of a reference standard, the microarray substrate includes reference-pattern features. Each reference-pattern feature further comprises a set of different probe molecules that bind to respective targets within a range of expected biological samples. For example, if the biological sample is derived from human tissue, then a set of different sequences complementary to Alu repeat sequences present in the biological sample may be attached to the positive reference-pattern features.

One method that employs a two-dimensional reference pattern, that represents one embodiment of the present invention, can be employed for quality control during the manufacturing process. First, a sample batch of manufactured microarrays can be exposed to a sample solution, scanned, initially processed, and imaged. The images include indications of the computed centers for the features within the pattern blocks of the reference patterns included in the microarrays. If the computed centers noticeably deviate from the feature centers in the reference pattern of a microarray, then the feature signals of the microarray may not be reliably extracted, or may include systematic errors of the types discussed above. In an alternative method, an automated feature-extraction system may use reference patterns to determine whether or not to proceed with feature extraction following initial processing steps, or what type of feature extractions methods should be employed, depending on how badly re-computed feature centers deviate from true feature centers. In alternative methods, users may employ visual inspection of reference patterns to monitor microarray quality following handling, storage, and experimental procedures.

Although the present invention has been described in terms of a particular embodiment, it is not intended that the invention be limited to this embodiment. Modifications within the spirit of the invention will be apparent to those skilled in the art. For example, as discussed above, the design of a two-dimensional reference pattern may be modified to include additional pattern blocks. Although a hexagonal arrangement of control features are illustrated throughout this disclosure to facilitate the discussion, other types of arrangements may suffer the above-discussed problems, and may be diagnosed for feature extractability by methods of the present invention. Although some problems causing centroid-displacement artifacts such as variability in feature size and differential signal intensities among adjacent features are specifically discussed above, a number of other types of variations, that may be introduced during the manufacturing process, and that result in difficulties in feature extraction, can be monitored by using these reference patterns as a calibration device during quality-control procedures. In an alternative embodiment, the reference pattern can be implemented as part of an automated feature extraction method so that, after initial feature finding using a rectilinear-coordinate system, the feature-extractability of the reference pattern can be determined. And almost limitless number of different embodiments are possible, depending on in what medium the method is implemented and on details of implementation. For example, embodiments may be implemented in hardware, software, firmware, or a combination of two or more of hardware, software, and firmware, and software or logic may have many different modular organizations, use any of different control and data structures, and, in the case of software implementations, may be written in any of numerous different programming languages.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A microarray comprising:
a substrate;
a number of features comprising probe molecules, each feature located at a different position on a surface of the substrate; and
at least one reference pattern that comprises a number of pattern blocks, each pattern block comprising an arrangement of one or more nearest-neighbor, positive-control features around a central feature, a comparison of computed positions for reference-pattern features to reference-pattern feature positions, following scanning of the microarray, indicating a feature extractability problem.

2. The microarray of claim 1 wherein the reference pattern includes a pattern block for each possible arrangement of one or more nearest-neighbor positive-control features around a central, positive-control feature and for each possible arrangement of one or more nearest-neighbor, positive-control features around a central, negative-control feature.

3. The microarray of claim 1 wherein the reference pattern comprises a two-dimensional array of pattern blocks.

4. The microarray of claim 1 wherein features are arranged in a hexagonal packing arrangement on the surface of the substrate, each interior feature having 4 nearest-neighbor features.

5. The microarray claim 1 wherein the reference pattern is positioned at one or more corners of the microarray.

6. The microarry of claim 1 wherein the reference pattern is positioned at one or more opposing diagonal corners of the microarray.

7. A kit comprising a microarray of claim 1.

8. The kit according to claim 7 wherein the microarray comprises reference-pattern features, each reference-pattern feature further comprising a set of different probe molecules that can bind to respective targets within a biological sample.

9. A kit comprising:
a microarray of claim 1; and
a set of reference targets that bind to the features of the pattern blocks when the microarray is exposed to the reference targets so that the features of the pattern blocks bound by the reference targets represent multiple different arrangements of one or more nearest neighbor positive control features around a central feature.

10. The kit according to claim 9 wherein the features of the pattern blocks bound by the reference targets represent all of the different arrangements of one or more nearest neighbors around a central feature.

11. The kit according to claim 9 further comprising instructions to expose the array to the reference targets.

12. A method for evaluating a microarray containing features at feature positions for feature extractability, the method comprising:
reading the microarray to produce a digital image of the microarray;
computing feature positions of one or more reference patterns within the microarray from the digital image;
comparing the computed reference-pattern-feature positions with reference-pattern-feature positions; and
when the computed reference-pattern-feature positions differ from the reference-pattern-feature positions by more than a threshold amount, determining the microarray to have a feature extractability problem.

13. The method of claim 12 wherein the reference pattern comprises at least one pattern block for each possible arrangement of one or more nearest-neighbor positive-control features around a central, positive-control feature.

14. The method of claim 12 wherein the reference pattern comprises at least one pattern block for each possible arrangement of one or more nearest-neighbor, positive-control features around a central, negative-control feature.

15. The method of claim 12 wherein the reference pattern comprises at least one pattern block for each possible arrangement of one or more nearest-neighbor positive-control features around a central, positive-control feature, and for each possible arrangement of one or more nearest-neighbor, positive-control features around a central, negative-control feature.

16. The method of claim 12 wherein comparing the computed reference-pattern-feature positions with reference-pattern-feature positions further includes visually comparing indications of the computed feature positions with images of the reference-pattern features.

17. The method of claim 12 wherein comparing the computed reference-pattern-feature positions with reference-pattern-feature positions further includes comparing, by a computer program, each computed feature position with the reference-pattern-feature positions.

18. The method of claim 12, wherein the method for evaluating a microarray for feature extractability can be carried out prior to full, feature extraction in order to determine whether or not full, feature extraction method can be conducted on the microarray.

19. The method of claim 12 further comprising:
fabricating a first batch of arrays;
reading a microarray selected from the first batch of arrays, to compute feature positions of one or more reference patterns, and comparing the computed reference-pattern-feature positions with reference-pattern-feature positions to determine whether or not the microarray has a feature extractability problem; and
rejecting other arrays of the batch when a feature extractability problem is determined.

20. The method according to claim 19 further comprising manufacturing a second batch of arrays and testing the second batch of arrays when the first batch of arrays is rejected.

21. A method according to claim 19 further comprising, when no feature extractability problem is determined, shipping other arrays of the batch to one or more remote locations along with an indication that features can be extracted using one or more referenced feature extraction methods.
